(19) United States
(12) Patent Application Publication
(10) Pub. No.: US 2016/0331817 A1
(43) Pub. Date: Nov. 17, 2016

(54) COMPOSITIONS AND METHODS FOR TREATING FIBROTIC MYOPATHY CONDITIONS USING COLLAGENASE

(71) Applicant: Raymond B. Raven, Pasadena, CA (US)

(72) Inventor: Raymond B. Raven, Pasadena, CA (US)

(21) Appl. No.: 14/710,015

(22) Filed: May 12, 2015

(51) Int. Cl. A61K 38/48

(52) U.S. Cl. CPC A61K 38/4886; C12Y 304/24003

(57) ABSTRACT

Compositions and methods for treating fibrotic myopathy in a subject.

### FIELD OF THE INVENTION

The present invention generally relates to compositions and methods for the treatment fibrotic myopathy in a subject. Specifically, the compositions and methods of the invention may be used to absorb damaged or scarred tissue associated with fibrosis.

### BACKGROUND OF THE INVENTION

Fibroblasts are the major cell type responsible for the synthesis of collagen, a fibrous protein essential for maintaining the integrity of the extracellular matrix found in the dermis of the skin and other connective tissues. The production of collagen is a finely regulated process, and its disturbance may lead to the development of tissue fibrosis. The formation of fibrous tissue is part of the normal healing process after injury, including injury due to infection or trauma.

In some circumstances, there is an abnormal accumulation of fibrous material such that it interferes with the normal function of the affected tissue. Additionally, fibrotic growth can proliferate and invade healthy surrounding tissue, even after the original injury heals. In some animals, formation of fibrous tissue is a chronic, progressive, idiopathic, degenerative disorder affecting muscles.

Abnormal accumulation of collagen in the extracellular matrix can cause fibrosis of a number of tissues including the skeletal muscles (fibrotic myopathy). The presence of fibrotic scar tissue in muscles results in altered muscle function, particularly with regard to pliability or stretchability, and typically imposes biomechanical restrictions on the affected muscle(s). Clinical signs include lameness, weakness of the affected limb, pain in the acute phases, decreased range of motion of the joints and firm scar tissue within the affected muscle. In the case of fibrotic myopathy, surgical release of affected tissues via tenotomy, myotenotomy, Z-plasty, or complete resection produces inconsistent results. Prognosis is guarded due to recurrence.

Therefore, there is a need for new simple and effective therapeutic methods to treat fibrotic myopathy without the need for surgical interference which is expensive, time consuming, potentially dangerous to the subject, and produces inconsistent results.

### SUMMARY OF THE INVENTION

The present invention is generally directed to the treatment and prevention of fibrotic myopathy in a subject. More particularly, the present invention advantageously provides a method for treating fibrotic myopathy in a companion animal by administering to the companion animal an effective amount of a composition comprising an anti-fibrotic agent.

The disclosure provides a method of treating fibrotic myopathy in a subject, the method comprising administering an effective amount of a composition comprising an anti-fibrotic agent to a fibrotic area of a muscle. The anti-fibrotic agent may be pancreatic elastase, elastase-2a, elastase-2b, neutrophil elastase, proteinase-3, endogenous vascular elastase, cathepsin G, mast cell chymase, mast cell tryptase, plasmin, thrombin, granzyme B, cathepsin S, cathepsin K, cathepsin L, cathepsin B, cathespin C, cathepsin H, cathespin F, cathepsin G, cathepsin O, cathepsin R, cathepsin V (cathepsin 12), cathepsin W, calpin 1, calpin 2, chondroitinase ABC, chondroitinase AC, hyaluronidase, chymopapain, chymotrypsin, legumain, cathepsin Z (cathepsin X), cathepsin D, cathepsin E, microbial collagenases, matrix metalloproteinases, ADAMTS-1, ADAMTS-2, ADAMTS-3, ADAMTS-4 (aggrecanase-1), ADAMTS-5(aggrecanase-2), ADAMTS-14, papain, subtilisin, subtilisin A, heparanase, and combinations thereof. In some aspects, the anti-fibrotic agent agent is a matrix metalloproteinase enzyme (MMP). In certain aspects, the MMP is collagenase derived from Clostridium histolyticum. In a preferred aspect, the collagenase derived from Clostridium histolyticum comprises collagenase I, collagenase II, and combinations thereof

In some aspects, the composition is administered in combination with one or more other therapeutics.

The disclosure further provides that the anti-fibrotic composition may be administered sub-dermally to the fibrotic area of a subject.

Other aspects and iterations of the invention are detailed below.

### DETAILED DESCRIPTION OF THE INVENTION

The present invention is directed to compositions and methods for treating fibrotic myopathy conditions in a subject, specifically in companion or domestic animals such as a dog, cat, or horse. More specifically, it has been discovered that fibrotic myopathy in companion or domestic animals can be treated by administering a composition comprising an anti-fibrotic agent at, near, or into fibrotic tissue causing the fibrotic myopathy. Accordingly, the present invention provides compositions capable of treating fibrotic myopathy, and methods of using the compositions to treat fibrotic myopathy in a companion animal.

### (a) Composition

In one aspect, the present invention provides compositions comprising an anti-fibrotic agent. Non-limiting examples of anti-fibrotic agents include pancreatic elastase, elastase-2a, elastase-2b, neutrophil elastase, proteinase-3, endogenous vascular elastase, cathepsin G, mast cell chymase, mast cell tryptase, plasmin, thrombin, granzyme B, cathepsin S, cathepsin K, cathepsin L, cathepsin B, cathespin C, cathepsin H, cathespin F, cathepsin G, cathepsin O, cathepsin R, cathepsin V (cathepsin 12), cathepsin W, calpin 1, calpin 2, chondroitinase ABC, chondroitinase AC, hyaluronidase, chymopapain, chymotrypsin, legumain, cathepsin Z (cathepsin X), cathepsin D, cathepsin E, microbial collagenases, matrix metalloproteinases, ADAMTS-1, ADAMTS-2, ADAMTS-3, ADAMTS-4 (aggrecanase-1), ADAMTS-5(aggrecanase-2), ADAMTS-14, papain, subtilisin, subtilisin A, heparanase, or a combination thereof.

In some embodiments, an anti-fibrotic agent of the invention is a collagenase. Preferably, an anti-fibrotic agent suitable for a composition of the present invention is a collagenase capable of digesting any type of collagen in fibrotic myopathy. In general, collagenases are matrix metalloproteinase enzymes (MMPs). MMPs are zinc-dependent endopeptidases capable of degrading a number of extracellular matrix proteins such as collagen, but also can process a number of bioactive molecules. Non limiting examples of collagenases include microbial collagenases, mammalian collagenases, and combinations thereof.

In some embodiments, an anti-fibrotic agent of the invention is a mammalian collagenase capable of digesting collagen. Non-limiting examples of a mammalian collagenase suitable for a composition of the present invention include mammalian MMPs such as MMP1, MMP2, MMP3, MMP7, MMP8, MMP9, MMP10, MMP11, MMP12, MMP13, MMP14, MMP15, MMP16, MMP17, and microbial MMPs. Preferably, a suitable mammalian collagenase may be MMP1, MMP2, MMP8, MMP9, MMP13, MMP14, MMP18, and combinations thereof.

In other embodiments, an anti-fibrotic agent of the invention is a microbial collagenase. Microbial collagenases assist in destroying extracellular structures in the pathogenesis of bacteria such as Vibrio and Clostridium. These collagenases are considered a virulence factor targeting the connective tissue in muscle cells and other body organs. In some embodiments, an anti-fibrotic agent of the invention is a collagenase derived from Vibrio bacteria.

In preferred embodiments, an anti-fibrotic agent of the invention is a collagenase derived from Clostridium bacteria. Culture filtrates of Clostridium histolyticum contain at least seven distinct collagenases with molecular weights that vary from 68,000 Da to 130,000 Da. These enzymes have been divided into two classes and are known as collagenase I and collagenase II. The terms “Collagenase I”, “ABC I”, “AUX I”, “collagenase AUX I”, and “collagenase ABC I” as used herein refer to the same enzyme and may be used interchangeably. Similarly, the terms “Collagenase II”, “ABC II”, “AUX II”, “collagenase AUX II”, and “collagenase ABC II” as used herein refer to the same enzyme and may be used interchangeably. The two classes of collagenases differ with respect to their sequences, as determined by the chromatographic analysis of their tryptic digests and cyanogen bromide reaction products, their mode of attack of native collagen, their relative activities toward synthetic peptides, and their differential inhibition of substrate analogs. Methods of deriving collagenases from Clostridium bacteria are known in the art and may be as described in U.S. Pat. No. 7,811,560, the disclosure of which is enclosed herein in its entirety. In general, collagenases are derived from Clostridium bacteria by fermenting Clostridium histolyticum, harvesting from the fermentation medium a crude product comprising collagenase I and collagenase II, and purifying collagenase I and collagenase II from the crude harvest.

When anti-fibrotic agents of a composition of the invention are collagenases derived from Clostridium bacteria, the anti-fibrotic agent may be collagenase I, collagenase II, or a combination of collagenase I and collagenase II. In one embodiment, an anti-fibrotic agent of the invention is collagenase I derived from Clostridium bacteria. In another embodiment, an anti-fibrotic agent of the invention is collagenase II derived from Clostridium bacteria. In a preferred embodiment, an anti-fibrotic agent of the invention is a combination of collagenase I and collagenase II derived from Clostridium bacteria.

When an anti-fibrotic agent is a combination of purified collagenase I and collagenase II, compositions of the invention may be prepared by mixing either specific masses of the purified collagenase enzymes or a specific number of activity units. In some embodiments, an anti-fibrotic agent is a combination of purified collagenase I and collagenase II, wherein collagenase I and collagenase II are combined by mixing a specific number of activity units. Collagenase activity may be measured by the enzyme's ability to hydrolyze either synthetic peptide or collagen substrate. Methods of measuring collagenase activity are known in the art.

In preferred embodiments, an anti-fibrotic agent is a combination of purified collagenase I and collagenase II, wherein collagenase I and collagenase II are combined by mixing specific masses of the purified collagenase enzymes. For instance, collagenase I and collagenase II may be mixed in a mass ratio range of about 0.1:100 to about 100:0.1. In a preferred alternative of the embodiments, an anti-fibrotic agent is collagenase I and collagenase II mixed in a mass ratio of about 1:1. Those skilled in the art will recognize that enzyme assays may also be used to define and prepare functionally equivalent enzyme compositions.

Compositions typically comprise therapeutically effective amounts of an anti-fibrotic agent. By a “therapeutically effective amount” of an anti-fibrotic agent is meant an amount of the agent which confers a therapeutic effect on the treated subject, at a reasonable benefit/risk ratio applicable to any medical treatment. A therapeutically effective amount of an anti-fibrotic agent can and will vary depending on the anti-fibrotic agent, the body weight, sex, age and/or medical condition of the subject, the severity and extent of the fibrotic myopathy in the subject, the method of administration, the duration of treatment, as well as the species of the subject, and may be determined experimentally using methods known in the art.

In certain embodiments, when the anti-fibrotic agent is collagenase, the collagenase is provided as a lyophilized product comprised of collagenase I and collagenase II in an approximate 1:1 mass ratio. The lyophilized collagenase product has a purity of about 99%, 95%, 80%, 75%, 70%, 65%, 60%, 55%, 50%, 45%, 40%, 35%, 30%, 25%, 20%, 15%, 10%, 5%, or 1%. Preferably, the lyophilized collagenase product has a purity of at least about 95% by area as determined by reverse phase high performance liquid chromatography (as described in, for example, in U.S. Pat. No. 7,811,560).

In certain embodiments, when the anti-fibrotic agent is collagenase, the concentration of collagenase in a composition may be about 0.1 mg/ml to about 10 mg/ml. Preferably, the concentration of collagenase present in the composition may range from between about 0.5 mg/ml to about 2 mg/ml of the total composition, preferably ranges from about 0.8 mg/ml to about 1.2 mg/ml.

In certain embodiments, when the anti-fibrotic agent is collagenase, the concentration of collagenase in a composition may be about 50 to about 100,000 ABC units/mi. For instance, the concentration of collagenase in a composition may be about 50, 100, 200, 300, 400, 500, 1000, 2000, 3000, 4000, 5000, 10,000, 20,000, 30,000, 40,000, 50,000, 60,000, 70,000, 80,000, 90,000, or about 100,000 ABC units/ml. Preferably, the concentration of collagenase present in the composition may range from between about 8000 to about 40,000 ABC units/ml of the total composition, preferably ranges from about 14,000 ABC units/ml to about 20,000 ABC units/ml.

Anti-fibrotic compositions of the present invention may be incorporated into pharmaceutical formulations suitable for in vivo, in vitro, in situ, or ex vivo use. The use of such media and agents for pharmaceutically active substances is well known in the art. Except insofar as any conventional media or agent is incompatible with a compound, use thereof in the compositions is contemplated. Supplementary active compounds may also be incorporated into the compositions.

A pharmaceutical composition of the invention may be formulated to be compatible with its intended route of administration. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Non-limiting examples of routes of administration include parenteral, e.g., intravenous, intradermal, subcutaneous, transdermal (topical), and transmucosal. Preferably, a pharmaceutical composition of the invention is formulated for parenteral administration. In preferred embodiments, a pharmaceutical composition is formulated for subcutaneous administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH may be adjusted with acids or bases, such as hydrochloric acid or sodium hydroxide. Parenteral preparations may be enclosed in ampoules, disposable syringes, or multiple dose vials made of glass or plastic.

Pharmaceutical compositions suitable for injectable use may include sterile aqueous solutions (where water soluble) or dispersions and sterile powders for the extemporaneous preparation of sterile injectable solutions or dispersion. Suitable carriers include physiological saline, bacteriostatic water, Cremophor EL (BASF; Parsippany, N.J.), or phosphate buffered saline (PBS). In all cases, a composition may be sterile and may be fluid to the extent that easy syringeability exists. A composition may be stable under the conditions of manufacture and storage and may be preserved against the contaminating action of microorganisms such as bacteria and fungi. The carrier may be a solvent or dispersion medium containing, for example, water, ethanol, polyol (for example, glycerol, propylene glycol, and liquid polyetheylene glycol, and the like), and suitable mixtures thereof. Prevention of the action of microorganisms may be achieved by various antibacterial and antifungal agents, for example, parabens, chlorobutanol, phenol, ascorbic acid, thimerosal, and the like. In many cases, it may be preferable to include isotonic agents, for example, sugars, polyalcohols such as mannitol, sorbitol, or sodium chloride, in the composition. Prolonged absorption of the injectable compositions may be brought about by including in the composition an agent which delays absorption, for example, aluminum monostearate and gelatin.

Sterile injectable solutions may be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying, which yields a powder of the active ingredient plus any additional desired ingredient from a previously sterile-filtered solution thereof.

Compounds may be prepared with carriers that will protect a compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylactic acid. These may be prepared according to methods known to those skilled in the art, for example, as described in U.S. Pat. No. 4,522,811.

### (b) Methods of the Invention

In another aspect, the invention encompasses a method of treating a connective tissue disorder in a subject. As described above, a preferred method of the invention may be used to treat fibrotic myopathy in a subject. A method of the present invention comprises administering a composition comprising an anti-fibrotic agent at, near, or into fibrotic tissue causing the connective tissue disorder. Preferably, a method of the invention comprises sub-dermal administration of a composition comprising an anti-fibrotic agent at or near fibrotic tissue causing the connective tissue disorder. Compositions comprising an anti-fibrotic agent may be as described in Section (a) above.

In some embodiments, a method of the invention may be used to treat fibrotic myopathy in companion animals, more specifically in dogs or horses. In a preferred embodiment, a method of the invention is used to treat fibrotic myopathy in dogs. Fibrotic myopathy in dogs may be a result of muscle injury. Alternatively, fibrotic myopathy in dogs may be a chronic, progressive, idiopathic, degenerative disorder affecting the semitendinosus, gracilis, quadriceps, infraspinatus, and supraspinatus muscles, primarily in dogs. Clinical signs of fibrotic myopathy in dogs include a nonpainful, mechanical lameness, wherein neurologic function is normal.

In other embodiments, a method of the invention may be used to treat fibrotic myopathy in horses. Fibrotic myopathy in horses describes a classic gait abnormality that develops when horses injure their semitendinosus and semimembranosus muscles at the point of a tendinous insertion during exercise that requires abrupt turns and sliding stops. Trauma (e.g., catching a foot in a fence), IM injections, and a congenital form are other potential causes of fibrotic myopathy. Affected muscles in acute cases are warm and painful on deep palpation. Chronically, hardened areas within the muscle may represent fibrosis and ossification. Chronically, fibrous replacement of muscle fibers may be apparent.

A method of the invention comprises administering one or more sub-dermal dose injections of a composition of the invention to an area affected by fibrotic myopathy. An area affected by fibrotic myopathy (also referred to herein as the “target area” or “treatment area”) is typically an area on one of the major muscle groups in an animal, such as the thighs and/or buttocks. An area affected by fibrotic myopathy may exhibit symptoms such as a discernable cord, dense muscle tissue, or contracted area of muscle. Preferably, a method of the invention comprises administering one or more sub-dermal doses may be administered where the dense muscle tissue or cord is most discernable.

As will be appreciated by one of skill in the art, a dose of a composition of the invention can and will vary depending on the body weight, sex, age and/or medical condition of the subject, the severity and extent of the fibrotic myopathy in the subject, the method of administration, and the duration of treatment, as well as the species of the subject. When an anti-fibrotic agent is collagenase, a typical dose of a composition of the invention for injection may comprise about 0.1 mg of collagenase to about 5 mg of collagenase per injection. In preferred embodiments, a dose of a composition of the invention for injection comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or about 5 mg of collagenase per injection. In a preferred alternative of the embodiments, a dose comprises about 0.3, 0.4, 0.5, 0.6, or about 0.7 mg of collagenase per injection. In another preferred alternative of the embodiments, a dose comprises about 1.8, 1.9, 2.0, 2.1, or about 2.3 mg of collagenase per injection.

When the subject is a dog, a dose of a composition of the invention may be about 0.1, 0.2, 0.3, 0.4, 0.5, 0.6, 0.7, 0.8, 0.9, to about 1 mg of collagenase per injection. In preferred embodiments when the subject is a dog, a dose of a composition of the invention is about 0.5 mg of collagenase per injection.

When the subject is a horse, a dose of a composition of the invention may be about 1, 1.5, 2, 2.5, or about 3 mg of collagenase per injection. In preferred embodiments when the subject is a horse, a dose of a composition of the invention is about 2 mg of collagenase per injection.

Alternatively, when an anti-fibrotic agent is collagenase, typical doses of a composition of the invention for injection may comprise about 1000 to about 100,000 ABC units of collagenase per injection. In preferred embodiments, doses of a composition of the invention for injection comprise about 1000, 5000, 10,000, 20,000, 30,000, 40,000, 50,000, 60,000, 70,000, 80,000, 90,000, or about 100,000 ABC units of collagenase per injection. In a preferred alternative of the embodiments, doses of a composition of the invention for injection comprise about a dose comprises about 5000, 7000, 9000, 10,000, or about 12000 mg of collagenase per injection.

When the subject is a dog, a dose of a composition of the invention may be about 2000, 4000, 5000, 7000, 8000, 10000, 12000, 14000, 15000, to about 17000 ABC units of collagenase per injection. In preferred embodiments when the subject is a dog, a dose of a composition of the invention is about 8000 ABC units of collagenase per injection.

When the subject is a horse, a dose of a composition of the invention may be about 8000, 25000, 35000, 45000, or about 50000 ABC units of collagenase per injection. In preferred embodiments when the subject is a horse, a dose of a composition of the invention is about 35000 ABC units of collagenase per injection.

The timing and duration of administration of the composition of the invention can and will vary. For instance, when a composition is administered to prevent fibrotic myopathy from developing, the composition may be administered shortly after an injury that may cause fibrotic myopathy to develop. Alternatively, a composition is administered after fibrotic myopathy has developed.

A composition may be administered at various intervals. The intervals can and will vary depending on the severity and extent of the fibrotic myopathy in the subject and may be determined experimentally using methods known in the art. For instance, a composition may be administered daily, weekly, monthly or over a number of months. In some embodiments, a composition is administered daily. In other embodiments, a composition is administered weekly. In yet other embodiments, a composition is administered monthly. In preferred embodiments, a composition is administered every three to six months. As it will be recognized in the art, the duration of treatment can and will vary depending on the progress of treatment.

### DEFINITIONS

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

As used herein, the term “anti-fibrotic agent” may refer to one or more chemical or biological compounds that degrades or causes the dissolution or shrinkage of fibrotic tissue or a portion thereof, such as for example, the fibrotic tissue in fibrotic myopathy. These compounds may have different mechanisms of action, some reducing the formation of fibrotic tissue, and others enhancing the metabolism or removal of fibrotic tissue in the affected area of the body. As such, an anti-fibrotic agent, includes, but is not limited to, an agent that degrades or causes the dissolution or shrinkage of fibrotic tissue or a portion thereof.

As used herein, the term “collagenase” may be used to describe any anti-fibrotic agent capable of digesting or causing the breakdown or dissolution of collagen. Collagen is the main structural protein of the various connective tissues in animals. In muscle tissue, collagen serves as a major component of the connective tissue. At least about 28 types of collagen have been identified.

As used herein, the language “pharmaceutically acceptable carrier” is intended to include any and all solvents, dispersion media, coatings, antibacterial and antifungal agents, isotonic and absorption delaying agents, and the like, compatible with pharmaceutical administration.

As used herein, the terms “treating,” “treatment,” or “to treat” each may mean to alleviate, suppress, repress, eliminate, prevent or slow the appearance of symptoms, clinical signs, or underlying pathology of a condition or disorder on a temporary or permanent basis. Preventing a condition or disorder involves administering an agent of the present invention to a subject prior to onset of the condition. Suppressing a condition or disorder involves administering an agent of the present invention to a subject after induction of the condition or disorder but before its clinical appearance. Repressing the condition or disorder involves administering an agent of the present invention to a subject after clinical appearance of the disease. Prophylactic treatment may reduce the risk of developing the condition and/or lessen its severity if the condition later develops. For instance, treatment of an existing fibrotic myopathy condition may reduce, ameliorate, or altogether eliminate the condition, or prevent it from worsening.

As used herein, the terms “about” and “approximately” designate that a value is within a statistically meaningful range. Such a range can be typically within 20%, more typically still within 10%, and even more typically within 5% of a given value or range. The allowable variation encompassed by the terms “about” and “approximately” depends on the particular system under study and can be readily appreciated by one of ordinary skill in the art.

As used herein, the term “w/w” designates the phrase “by weight and is used to describe the concentration of a particular substance in a mixture or solution.

As used herein, the term “mL/kg” designates milliliters of composition per kilogram of body weight.

As used herein, the term “subject” refers to an animal. The subject may be an embryo, a juvenile, or an adult. Suitable subjects include vertebrates such as mammals, birds, reptiles, amphibians, and fish. Examples of suitable mammals include, without limit, rodents, companion or domestic animals, livestock, and primates. Non-limiting examples of rodents include mice, rats, hamsters, gerbils, and guinea pigs. Non-limiting examples of livestock include goats, sheep, swine, cattle, llamas, and alpacas. Suitable primates include, but are not limited to, humans, capuchin monkeys, chimpanzees, lemurs, macaques, marmosets, tamarins, spider monkeys, squirrel monkeys, and vervet monkeys. Non-limiting examples of birds include chickens, turkeys, ducks, and geese.

As used herein, the terms “companion animal” or “domestic animal” refer to an animal typically kept as a pet for keeping in the vicinity of a home or domestic environment for company or protection, regardless of whether the animal is kept indoors or outdoors. Non-limiting examples of companion animals or domestic animals include, but are not limited to, dogs, cats, house rabbits, ferrets, and horses. An exemplary subject is a dog. Another exemplary subject is a horse.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” in some embodiments denotes that a protein gives rise to essentially one band in an electrophoretic gel. Preferably, it means that the protein is at least 85% pure, more preferably at least 95% pure. “Purify” or “purification” in other embodiments means removing at least one contaminant from the composition to be purified. In this sense, purification does not require that the purified compound be homogenous, e.g., 100% pure.

The following examples are intended to further illustrate and explain the present invention. The invention, therefore, should not be limited to any of the details in these examples.

### EXAMPLES

### Example 1

Treating muscular myopathy in dogs.

A collagenase preparation comprising two collagenases, collagenase I and collagenase II from Clostridium histolyticum may be used in this study. The ollagenase I and collagenase II may be present at an approximate mass ratio of 1:1. The lyophilized preparation may be reconstituted in a solution to generate a preparation. The preparation may be sterile-filtered before administration. Each dose for administration may be about 0.5 mg/ml collagenase.

The collagense preparations may be administered to dogs exhibiting symptoms of fibrotic myopathy in muscles such as the semitendinosus, gracilis, quadriceps, infraspinatus, and supraspinatus muscles. As a control, the collagense preparations may also be administered to healthy dogs. Each dog may receive one dose in a muscle affected by muscular myopathy. The dose may be administered throughout the affected muscle.

The severity of the muscular myopathy symptoms may be monitored over the course of about 3 to about 6 months. Additional subcutaneous injections may be administered as needed. In some cases, physical therapy may also be administered during the treatment period.

### EXAMPLE 2

Treating muscular myopathy in horses.

A collagenase preparation comprising two collagenases, collagenase I and collagenase II from Clostridium histolyticum may be used in this study. The ollagenase I and collagenase II may be present at an approximate mass ratio of 1:1. The lyophilized preparation may be reconstituted in a solution to generate a preparation. The preparation may be sterile-filtered before administration. Each dose for administration may be about 2 mg/ml collagenase.

The collagense preparations may be administered to horses exhibiting symptoms of fibrotic myopathy in muscles. As a control, the collagense preparations may also be administered to healthy horses. Each horse may receive one dose in a muscle affected by muscular myopathy. The dose may be administered throughout the affected muscle.

The severity of the muscular myopathy symptoms may be monitored over the course of about 3 to about 6 months. Additional subcutaneous injections may be administered as needed. In some cases, physical therapy may also be administered during the treatment period.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the following claims.

1. A method of treating fibrotic myopathy in a subject, the method comprising the step of:
administering an effective amount of a composition comprising collagenase to a fibrotic area of a muscle of the subject,
wherein
the composition is administered sub-dermally to the fibrotic area in at least one injection.

2. The method according to claim 1, wherein the anti-fibrotic agent is selected from the group consisting of pancreatic elastase, elastase-2a, elastase-2b, neutrophil elastase, proteinase-3, endogenous vascular elastase, cathepsin G, mast cell chymase, mast cell tryptase, plasmin, thrombin, granzyme B, cathepsin S, cathepsin K, cathepsin L, cathepsin B, cathespin C, cathepsin H, cathespin F, cathepsin G, cathepsin O, cathepsin R, cathepsin V (cathepsin 12), cathepsin W, calpin 1, calpin 2, chondroitinase ABC, chondroitinase AC, hyaluronidase, chymopapain, chymotrypsin, legumain, cathepsin Z (cathepsin X), cathepsin D, cathepsin E, microbial collagenases, matrix metalloproteinases, ADAMTS-1, ADAMTS-2, ADAMTS-3, ADAMTS-4 (aggrecanase-1), ADAMTS-5(aggrecanase-2), ADAMTS-14, papain, subtilisin, subtilisin A, heparanase, and combinations thereof.

3. The method according to claim 1,
wherein the collagenase selected from the group consisting of microbial collagenases, mammalian collagenases, and combinations thereof.

4. The method according to claim 3,
wherein the collagenase is derived from Clostridium histolyticum.

5. The method according to claim 4,
wherein the collagenase derived from Clostridium histolyticum comprises collagenase I, collagenase II, and combinations thereof.

6. The method according to claim 5,
wherein the collagenase I and collagenase II are present at a 1:1 mass ratio.

7. The method according to claim 4,
wherein the collagenase is a lyophilized collagenase product with a purity of at least about 95%.

8. The method according to claim 1,
wherein the concentration of anti-fibrotic agent in the composition ranges from about 0.8 mg/ml to about 1.2 mg/ml.

9. The method according to claim 1,
wherein a dose of the composition comprises about 0.5 mg of anti-fibrotic agent.

10. The method according to claim 1,
wherein a dose of the composition comprises about 2 mg of anti-fibrotic agent.

11. (canceled)

12. (canceled)

13. The method according to claim 1,
wherein the anti-fibrotic agent is administered at multiple sites to the fibrotic area.

14. The method of claim 1,
wherein treating fibrotic myopathy disorder cures the disorder, reduces the severity of the disorder, or ameliorates one or more symptoms of the disorder.

15. The method of claim 11,
wherein the anti-fibrotic agent degrades, shrinks, relaxes or stretches at least a portion of fibrotic tissue of the muscle.

16. The method of claim 1,
wherein the anti-fibrotic agent is administered in combination with one or more other therapeutics.

17. The method according to claim 1,
wherein the subject is a companion animal.

18. The method according to claim 17,
wherein the companion animal is selected from the group consisting of a horse and a dog.

19. A method of treating fibrotic myopathy in a subject, the method comprising the steps of:
a. identifying the subject having fibrotic myopathy; and
b. administering an effective amount of a composition comprising collagenase to a fibrotic area of a muscle of the subject,
wherein
the composition is administered sub-dermally to the fibrotic area in at least one injection.

20. A method of treating fibrotic myopathy in a subject, the method comprising the steps of:
a. identifying the subject having fibrotic myopathy; and
b. administering an effective amount of a composition comprising an anti-fibrotic agent to a fibrotic area of a muscle of a subject,
wherein,
the anti-fibrotic agent is a mammalian collagenase.
